(19) United States
(12) Patent Application Publication
(10) Pub. No.: US 2012/0003746 A1
(43) Pub. Date: Jan. 5, 2012

(54) METHOD AND KIT FOR IDENTIFICATION OF AN EXPLOSIVE SUBSTANCE WHICH CONTAINS AN OXIDANT

(75) Inventor: Shai Amisar, Tel Aviv (IL)

(73) Assignee: MISTRAL DETECTION LTD., HERZLIYA PITUACH (IL)

(21) Appl. No.: 13/146,261

(22) PCT Filed: Feb. 1, 2010
(86) PCT No.: PCT/IB10/50430

§ 371 Date: Jul. 26, 2011

Related U.S. Application Data
(60) Provisional application No. 61/202,132, filed on Jan. 30, 2009.

(51) Int. Cl. G01N 33/22; C07D 279/20

(52) U.S. Cl. 436/110; 436/124; 436/106; 544/37

(57) ABSTRACT

Field test kits for detecting explosive substances including a combination of reagents for sequentially testing for perchlorates, nitro aromatic compounds, nitroamines, nitroesters, chlorates, bromates, peroxides and nitrates are discussed. The reagents in the kits may contain methylene blue, methylene blue in a basic solution, and an acidified aromatic amine solution. Methods for employing the test kits are discussed, the methods being suitably adapted to detect the moieties suspected of being present. The kits may include a reagent for detecting explosive perchlorates, the reagent being methylene blue. The kits also include a reagent for detecting explosive nitroamines and nitroesters, the reagent being an acidified aromatic amine solution. The aromatic amine solution also can detect nitro aromatic explosives after a strong basic hydroxide has first been applied to the suspected substance.

### CROSS-REFERENCE TO RELATED APPLICATION

This application claims the benefit of priority from U.S. Provisional Application Ser. No. 61/202,132, filed Jan. 30, 2009, titled “Method for Identification of an Explosive Substance which Contains an Oxidant”.

### FIELD OF THE INVENTION

The present invention relates to an improved method and kit for detecting explosives selected from perchlorates, peroxides, chlorates, bromates, nitrates, nitro amines, nitro esters and/or nitro aromatics.

### BACKGROUND OF THE INVENTION

In recent times, the use of home-made, improvised explosives has been growing rapidly, and peroxide based explosives, like triacetonetriperoxide (TATP) and hexamethylenetriperoxidediamine (HMTD), have been shown to be easily manufactured and almost as strong as standard explosives. Moreover, some recent terror acts used rockets utilizing perchlorate solid fuel and home made explosives based on pyrotechnic devices containing perchlorates.

Methods and test kits for detecting explosives selected from nitro aromatics, organic nitrates, nitro amines, inorganic nitrates, chlorates and bromates, have been described by Margalit in U.S. Pat. Nos. 5,296,380 and 5,480,612. Neither of these patents describes detection of perchlorate-based explosives and both require a different step to detect nitrates that are used in home-based explosives. The entire contents of U.S. Pat. Nos. 5,296,380 and 5,480,612 are incorporated by reference herein.

Itzhaky et al., in WO99/043846, has described a method and kit for detecting an organic peroxide-based explosive in a sample. The organic peroxide is hydrolyzed with strong acid to release hydrogen peroxide, and the resulting mixture is reacted with a peroxidase enzyme and a substrate capable of being oxidized by the oxidant under catalysis by the enzyme to produce a pronounced change in a measurable physical parameter of the substrate. The entire contents of WO9943846 are incorporated by reference herein.

### SUMMARY OF THE PRESENT INVENTION

It is an object of the present invention to enable the detection of explosives containing perchlorates, chlorates and/or bromates.

It is a further object of the invention to provide for the sequential detection of peroxide-based explosives after testing for the presence of perchlorates, nitro aromatics, nitro amines, nitro esters, chlorates and bromates, in the same sample. After testing for peroxides testing for nitrates follows.

It is yet a further object of the invention to provide kits for such detection testing. In another object of the invention a novel reagent for perchlorate detection is provided.

Another object of the invention is to provide a novel reagent and procedure for determining the presence of nitro amine and/or nitro ester explosive substances after the nitro compounds have been treated with base.

In a further object of the invention methods for determining inter alia the above mentioned explosive materials are described.

Other objects of the invention will be apparent from the description which follows.

In a first aspect of the present invention there is provided a test kit for detecting an oxidant in a sample suspected to be an explosive, the kit comprising a methylene blue solution for detecting perchlorates and an absorbent medium for absorbing the methylene blue thereby enhancing visibility of any color change.

In an embodiment of the test kit, the kit comprises a methylene blue solution for detecting perchlorates and an aromatic amine solution that is highly acidified for detecting chlorates, bromates and/or nitrates.

In another embodiment of the test kit the kit comprises: a methylene blue solution for detecting perchlorates; a highly acidified solution containing an aromatic amine for detecting chlorates, bromates and/or nitrates and a solution of a strong base for determining the presence of a nitro aromatic compound. Contacting a sample with the solution of the strong base followed by contacting the same spot on the sample that was contacted previously with the highly acidified aromatic amine solution will reveal the presence of nitro amines and nitro esters.

In still another embodiment of the test kit, the kit comprises: a methylene blue solution for detecting perchlorates; an aromatic amine solution that is highly acidified for detecting chlorates, bromates and/or nitrates; a solution of a strong base for determining the presence of a nitro aromatic compounds; and one or more of 1. a solution of cations of a transition metal for detecting peroxides and 2. a reagent comprising zinc powder suspended in a liquid to detect nitrates.

In a further embodiment of the test kit, the kit comprises: a methylene blue solution for detecting perchlorates; an aromatic amine solution that is highly acidified for detecting chlorates, bromates and/or nitrates; a solution of a strong base for determining the presence of a nitro aromatic compound; one or more of 1. a solution of cations of a transition metal for detecting peroxides and 2. a reagent comprising zinc powder suspended in a liquid to detect nitrates; and a pH sensitive reagent for discriminating between alkali nitrates and amine nitrates when nitrates have been detected.

In another embodiment of the test kit, in addition to a methylene blue solution, the kit may comprise at least two of the following: a reagent comprised of an aromatic amine in a strongly, that is highly, acidified solution; a reagent comprising a solution of cations of a transition metal for detecting peroxides; a reagent comprising zinc powder suspended in a liquid; and a reagent comprising a solution of a strong base for inter alia forming a Meisenheimer complex with a nitro aromatic compound.

In an embodiment of a second test kit there is provided an aromatic amine in a highly acidified solution for detecting chlorates, bromates and/or nitrates.

In a further embodiment of the second test kit there is provided an aromatic amine in a highly acidified solution for detecting chlorates, bromates and/or nitrates, and a solution of a strong base for detecting a nitro aromatic compound.

In yet a further embodiment of the second test kit there is provided an aromatic amine in a highly acidified solution for detecting chlorates, bromates and/or nitrates, a solution of a strong base for detecting a nitro aromatic compound, and a methylene blue solution for detecting a perchlorate.

In all of the above test kits, the kits will include one or more dispensing devices selected from a spray device, a dropper device or breakable ampoules. Any of the above kits may be further characterized by one of more of the following features:

(a) one or more of the methylene blue and acidified aromatic amine reagents includes one or more water-miscible non-aqueous solvents;

(b) the aromatic amine comprises a carbocyclic secondary aromatic amine;

(c) the highly acidified solution containing an aromatic amine comprises sulfuric acid or phosphoric acid;

(d) the cations of the transition metal are cations selected from a group consisting of iron, copper, manganese, chromium, cobalt and ruthenium cations;

(e) the kit comprises an absorbent medium for sampling a suspected source of an explosive, the absorbent medium impregnated with a basic substance to neutralize excess acid in the acidified aromatic amine solution wherein the basic substance may be selected from a group consisting of sodium carbonate, sodium phosphate, potassium phosphate and sodium borate;

(f) the kit comprises an additional reagent adapted for testing for the presence of urea and nitro amines; and

(g) the kit comprises a reagent solution which is a strongly basic solution comprised of an alkali hydroxide or an alkyl ammonium hydroxide.

The methylene blue solutions in the above kits may comprise methylene blue in a water-based solution with a preferred concentration range of about 0.01% to about 0.2% w/w. The water-based solvent of the methylene blue solution may also contain a water miscible solvent. A typical such solvent is water and a water-miscible alcohol where the methylene blue is in a concentration range of about 0.01% to about 0.2% w/w and the alcohol is in a concentration range of about 10% to about 30% v/v alcohol to water.

The reagent for forming the Meisenheimer complex may be a strongly basic solution where the strongly basic solution is a solution comprising an alkali hydroxide or an alkyl ammonium hydroxides. In other instances, the reagent for forming the Meisenheimer complex is the methylene blue solution which further comprises a basic compound selected from a group consisting of an alkyl ammonium hydroxide, sodium hydroxide and potassium hydroxide.

In another aspect of the present invention there is provided a method for detecting and identifying explosives in a suspect substance. The method comprises the steps of: providing a sample of the suspect substance on an absorbent medium; contacting the sample with a solution for the detection of perchlorate, wherein if perchlorate is present the solution changes color; if no color change occurs, contacting a second sample with a strongly basic solution comprised of a base selected from an alkali hydroxide or a tetraalkylammonium hydroxide, the basic solution forming in the presence of aromatic nitro compounds a Meisenheimer complex having an identifiable color; placing a portion of a strongly acidified solution of an aromatic amine on substantially the same spot of the absorbent medium as in the previous step, the aromatic amine being oxidized by chlorates, bromates and nitrites, the latter released by the basic hydrolysis of nitro amines or nitro esters occurring in the previous step of contacting, thereby producing a change in color identifying the presence of nitro amines or nitro esters, chlorates, and bromates in the sample; when no reaction from the steps of contacting and the step of placing has occurred, placing a drop of solution containing transition metal cations in a non-aqueous or aqueous solvent on the same spot of the absorbent medium as in the previous steps and if peroxides are present, the cations catalyze oxidation of the aromatic amine to produce a blue color; and when no color change has occurred in any of the previous steps, applying a suspension of zinc dust to the same spot of the absorbent medium that under the acidic conditions resulting from the previous use of the acidified aromatic amine reagent reduced any nitrate salts present to nitrites that in turn oxidizes the aromatic amine causing the reagents to turn blue.

In the method, the aromatic amine is selected from a group consisting of diphenylamine, aniline, and tetramethyl diamino benzidine. The transition metal cations are selected from ions of the group of metals consisting of iron, copper, manganese, chromium, cobalt and ruthenium. The method is further characterized by one or more of the following features: (i) the water-based reagent includes one or more water-miscible non-aqueous solvents; (ii) the transition metal ions are ferric ions; and (iii) the perchlorate detecting reagent includes methylene blue.

In another embodiment of the method, the method may include the steps of: providing a sample of a substance suspected of being an explosive; contacting the sample of the substance with a first reagent, the first reagent being a solution of methylene blue which turns pink in the presence of perchlorate ion; contacting the same sample with a second reagent, the second reagent comprising an aromatic amine in a strongly acidic solution, whereby the presence of a chlorate or bromate explosive produces a distinct coloration; in the absence of such distinct coloration, contacting the same sample, which is already in contact with the first and second reagent, with a third reagent, the third reagent comprising a solution of cations of a transition metal, whereby the presence of organic peroxides in the sample, which has been hydrolyzed at least partly to hydrogen peroxide by the strong acid of the second reagent, produces a distinct coloration; and in the absence of any coloration, contacting the same sample, which is already in contact with the first, second and third reagents, with a fourth reagent, the fourth reagent comprising powdered zinc suspended in a liquid, and the fourth reagent producing a distinct blue color when nitrate ion is present.

The method may be further characterized by one or more of the following features: (a) the aromatic amine comprises a carbocyclic secondary aromatic amine; (b) the strong acid of the second reagent comprises sulfuric acid or phosphoric acid; and (c) the cations of the transition metal are cations of a metal selected from a group of metals consisting of iron, copper, manganese, chromium, cobalt and ruthenium. The method may be further characterized by one or more of the following features: (a) one or more of the first and second reagents includes one or more water-miscible non-aqueous solvents selected from the group consisting of dimethyl sulfoxide, dimethylformamide, dimethylacetamide, hexamethylphosphoramide, N-methylpyrrolidone and water-miscible alcohols and ethers; and (b) the aromatic amine comprises a diarylamine. The methylene blue solution may be a water-based solution. The water-miscible alcohol may be isopropyl alcohol, preferably in the concentration range of about 10% to about 30% v/v alcohol to water.

In yet another embodiment of the method, when nitrates are detected in a previous step of any of the previous embodiments of the method, the method may further include the step of: contacting the suspected explosive with a pH sensitive reagent for discriminating between alkali nitrate salts and amine nitrate salts and noting the pH, low pH indicating the possible presence of an explosive amine nitrate. The pH sensitive agent is selected from a group of pH indicators which undergo a color change below pH 5.

In still another embodiment of the method, the method is adapted for detecting perchlorate, nitro aromatic, nitro ester and nitrate explosives in a suspect substance, the method comprising the steps of: providing a sample of the suspect substance on an absorbent medium; contacting the sample with a solution for the detection of perchlorate wherein if perchlorate is present the solution changes color; contacting a sample with a solution of a strong base, wherein the base is selected from an alkali hydroxide or alkylammonium hydroxide, the basic solution forming, in the presence of aromatic nitro compounds, a colored Meisenheimer complex having an identifiable color change and if there are nitro amines or nitro esters present, the basic solution hydrolyzes the nitro amines or nitro esters to nitrites; placing a portion of a highly acidified solution of an aromatic amine on the same spot of the absorbent medium as in the previous step so that any nitrites present will oxidize the aromatic amine producing a change in color. The reagent for detecting perchlorates may be a methylene blue solution.

In yet another embodiment of the method, the method is adapted for detecting perchlorate, nitro aromatic, nitro ester, nitro amine and nitrate explosives, the method including the steps of: providing a sample of a suspect substance; contacting the sample with a reagent for detecting perchlorates; when there is no change in color in the step of contacting, contacting the same sample with a reagent for forming a Meisenheimer complex with a nitro aromatic compound, the reagent being a highly basic solution; when there is no reaction in the second step of contacting, contacting the same sample with a reagent that reacts with the nitrites generated by hydrolyzing nitro amines and nitro esters; and when there is no reaction, contacting the same sample with a reagent that reduces nitrates to nitrites.

The reagent to reduce nitrates to nitrites may be selected from powdered metals, preferably, zinc dust. In other instances, the reagent that reacts with nitro amines and nitro esters to hydrolyze their nitrogen moieties to nitrites may be selected from a group of reagents consisting of Greiss's reagent or an acidified aromatic amine solution. The reagent for detecting perchlorates may be a methylene blue solution.

In a further embodiment of the method of the invention there is provided a method for detection and identification of oxidant materials comprising the steps of: providing a first sample of a suspected substance; contacting the first sample with a solution of methylene blue to produce a color change in the presence of perchlorates; and reacting a second sample with a strongly acidified solution of aromatic amines to produce a color change in the presence of oxidants selected from the group of chlorates, bromates, permanganates, nitrates and chromates.

In yet another aspect of the present invention there is provided a reagent for the detection of perchlorate, the reagent comprising methylene blue.

In still another aspect of the present invention there is provided a reagent for the detection of nitro esters and nitro amines, the reagent comprising a highly acidified solution of an aromatic amine, wherein the reagent produces a change in color after reacting with nitrites resulting from the hydrolysis of nitro amines and nitro esters being exposed to a solution of a strong base. In some instances, the aromatic amine is a secondary carbocyclic amine. In some instances, the secondary carbocyclic amine is diphenylamine. In some instances, the reagent is acidified using concentrated sulfuric or phosphoric acid.

In a further aspect of the present invention, there is provided a reagent for the detection of nitrates comprised of highly acidified aromatic amine solution. The reagent may be further characterized by one or more of the following: (a) the acidification is effected by sulfuric acid; (b) the sulfuric acid is more that 50% w/w; and (c) the acidified solution contains less than 10% w/w alcohol.

### BRIEF DESCRIPTION OF THE DRAWING

The invention is herein described, by way of example only, with reference to the accompanying drawing. With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in greater detail than is necessary for a fundamental understanding of the invention. The description taken with the drawing makes apparent to those skilled in the art how the invention may be embodied in practice.

FIG. 1 is a flowchart of Method D based on Method A of the present invention as described herein.

FIG. 2 is a flowchart of Method D based on Method B of the present invention as described herein.

### DESCRIPTION OF PREFERRED EMBODIMENTS

The present invention provides simple, quick, field test kits for determining if suspected substances contain explosive materials. The materials may belong to explosive classes which contain perchlorates, chlorates, bromates, nitrates, peroxides, nitro amines, nitro esters, and nitro aromatics. The kits can also be used to test materials that may contain urea compounds such as urea nitrate. The kits are readily usable under field conditions by police departments, immigration services and other like institutions.

The present invention also provides a novel reagent for detecting perchlorates, the reagent containing methylene blue. The perchlorates that may be detected may be, but do not necessarily have to be, explosive perchlorate compounds. One methylene blue composition that can be prepared and used to detect perchlorates is described herein below.

The methylene blue reagent described herein may be dissolved in 1. water; or 2. in at least one water-miscible non-aqueous solvent, such non-aqueous solvent being at least one solvent selected from the group consisting of dimethyl sulfoxide, dimethylformamide, dimethylacetamide, hexamethylphosphoramide, N-methylpyrrolidone and water-miscible alcohols and ethers; or 3. a water/water miscible non-aqueous mixed solvent where the non-aqueous solvent is selected from the group listed in 2 above; or 4. any of the solvents in 1, 2, and 3 above to which a basic substance has been added. Without limiting the choice of the basic substance, the substance may be an alkali hydroxide or an alkyl ammonium hydroxide.

The present invention further provides a second novel reagent, which detects nitro amines and nitro esters. The reagent for this determination is a highly acidified solution of a primary, secondary or tertiary aromatic amine, typically, but without intending to limit the invention, a secondary aromatic amine such as a diarylamine. One preferable secondary aromatic amine comprises the carbocyclic secondary aromatic amine diphenylamine. Without limiting the invention, other aromatic amines can be used such as aniline or tetramethyl diaminobenzidine. Acidification of the solution may be carried out using sulfuric acid. Other strong acids such as phosphoric acid may also be used.

This highly acidified aromatic amine reagent is used after a suspected substance has been contacted with a solution of a strong base which converts the nitrogen moieties of the suspected substance to nitrites. In some instances the strongly basic solution may be a basic methylene blue solution. If perchlorate is present, the methylene blue will change to pink. If there are nitro aromatic compounds present, after exposure to the basic methylene blue solution or to a strongly basic solution, the strongly basic solution will cause the nitro aromatic compound to change to another distinctive color as a result of the formation of a Meisenheimer complex. When a strongly basic solution is used, it may be comprised of an alkali hydroxide or an alkyl ammonium hydroxide.

After exposure of the suspected substance to a strongly basic solution or a strongly basic solution of methylene blue, the nitro moieties of any nitro amines and nitro esters present are converted to nitrites. The nitrites react with the highly acidified aromatic amine reagent to form a colored oxidized amine.

The highly acidified aromatic amine reagent for determining the presence of nitro amines and nitro esters may also be used to determine the presence of nitrates. In the method of detecting nitrates a basic solution need not be used. When preparing the acidified aromatic amine reagent for use in detecting nitrates, it may be prepared in a solvent that inter alia contains alcohol. The alcohol concentration, however, should be kept low as higher alcohol concentrations slow down the reaction so that its advantage as a field reagent is reduced. In lieu of alcohol as a solvent or mixed water/alcohol solvent, a water miscible non-aqueous solvent other than alcohol may be used. Typical, non-aqueous solvents that may be used are dimethyl sulfoxide, dimethylformamide, dimethylacetamide, hexamethylphosphoramide, N-methylpyrrolidone and water-miscible ethers.

In the presence of chlorates, bromates, nitrates or other oxidants, the highly acidified aromatic amine reagent discussed herein above will be oxidized forming a colored reaction product indicative of such oxidants.

In the detection tests for nitro aromatics, nitro esters, nitro amines, nitrates, chlorates, bromates or other oxidants, the exact colors produced are dependent on the specific aromatic amine used in the acidified aromatic amine test reagent.

Use of a secondary aromatic amine, and in particular, diphenylamine, is presently preferred because it is much less toxic and more sensitive for detection and identification purposes than other secondary aromatic amines or aniline. Nevertheless, use of a primary amine such as aniline and other secondary and tertiary aromatic amines is also to be considered within the scope of the present invention.

The present invention also teaches methods for using the field test kits and the novel reagents described herein.

The main difference between the test kits of the present invention and competitor kits is the ability to detect and distinguish between a large variety of explosive materials and precursors in a minimum number of steps.

The present preferred embodiments will be discussed in the context of detecting and identifying explosives. It should readily be understood by one skilled in the art that, at most, with relatively small modifications the kits, the reagents, and the methods discussed herein may be appropriate for use with other chemical substances having chemical structures similar to those of the explosive substances discussed herein.

Before explaining at least one embodiment of the invention in detail, it is to be understood that the invention is not limited in its application to the details and the arrangement of the reagents and kits set forth in the following description. The invention is capable of other embodiments or of being practiced or carried out in various ways. Also, it is to be understood that the phraseology and terminology employed herein is for the purpose of description and should not be regarded as limiting.

In a preferred embodiment of the invention, the reagents which are used in the test kits taught herein may be prepared as follows.

Reagent A (for Perchlorates)

A solution of 0.01-0.5% w/v solution of methylene blue, preferably 0.01-0.05 in 20/80 mixture by volume of isopropyl alcohol and water is prepared.

It should be noted that the methylene blue reagent may be used as a detecting reagent for perchlorates alone or within a larger detection scheme useful for detecting other explosive constituents as well.

Reagent B (for Chlorates, Bromates or Nitrates)

A mixture of Reagent B is prepared by mixing 95% sulfuric acid 20-70% w/w with dimethyl sulfoxide (DMSO) 10-30%, preferably 20%, isopropanol alcohol (IPA) 0-30%, preferably 10%, water 10-50%, preferably 20%, and diphenylamine 0.01-1%, preferably 0.1% with stirring, until a homogeneous solution is obtained. The isopropyl alcohol (IPA) is used to increase solubility of the amine in the mixture. This reagent gives a deep blue coloration with chlorates or bromates within 1-2 seconds, which fades on standing. The reagent is sensitive to as little as 0.0000001 g/mm² of chlorate or bromate. Perchlorate does not give a positive reaction.

The amount of alcohol in the mix affects the reactivity of the reagent with nitrates. If the alcohol content of the solvent is reduced to under 10% the nitrate reaction is relatively quick and suitable for use as a field detection reagent. With more concentrated IPA solutions the reagent will react very slowly (5 minutes or more) with nitrates.

When used herein, Reagent B may be described as a “highly acidified aromatic amine” or a “strongly acidified aromatic amine” or a “highly acidic aromatic amine” or a “strongly acidic aromatic amine” or such similar terms. There is no intention at differentiating between these terms. The aromatic amine solutions referred to are prepared substantially as described in Reagent B above and are generally acidified with strong acids such as sulfuric acid and phosphoric acid.

Reagent C (for Organic Peroxides)

A solution of 0.01-1% FeCl₃, preferably 0.4-0.6% in an organic solvent such as DMSO or organic ethers, preferably dipropyleneglycol dimethyl ether. The sensitivity of this reagent is similar to that of reagent B above.

Reagent D (for Nitrates when Acid Concentration is Low or Required for the Differentiation Between Chlorates and Nitrates)

A suspension of zinc dust in an organic solvent like isopropyl alcohol and/or isobutanol and may contain a silica-based suspension aid.

It should be noted that reagent D (zinc dust) may be used to detect nitrates at low acid concentrations while detection of nitrates using reagent B requires highly acidic concentrations (and low alcohol concentrations) if the mixture is to be effective. Zinc dust is used to differentiate between chlorates and nitrates which may react similarly at an earlier stage of the detection sequence.

It should readily be understood by persons skilled in the art that other powdered metals may be used if their electrochemical properties are suitable. However, zinc dust is a preferred powdered metal discriminating between chlorates and nitrates.

Reagent E (for Nitro Aromatics, Nitro Amines, Nitro Esters)

This reagent is a basic solution usable either for the detection of nitro aromatic compounds or the hydrolysis of nitro esters or nitro amines. The nitro compounds are hydrolyzed to nitrites which in turn react with the highly acidified amine solution, i.e. Reagent B. Reagent E is a solution of alkyl ammonium hydroxide or alkali hydroxide, which can be used at concentrations of between about 0.1% to about 50%. The basic hydroxide compounds can be dissolved 1. in water, 2. in a non-aqueous solvent such as dimethyl sulfoxide (DMSO) or methanol, or 3. in a mixture of water with a non-aqueous solvent such as those mentioned in 2 above, or 4. in a mixture of water and a water miscible solvent like methanol, dimethyl sulfoxide (DMSO) and others.

Field test kits as contemplated by the present invention comprise any of the above described reagents prepared substantially as described above. All of the starting materials in the above preparation are readily available commercially from many different suppliers.

In addition to the reagents the preparation of which have been outlined above, the kit may have at least one additional reagent adapted for differentiating between at least one further substance in the suspect sample selected from urea nitrate and ammonium nitrate. One such reagent that may be added in the kit is Nessler's reagent which is usable for detecting ammonium ion. Typically, Nessler's reagent is prepared from potassium iodide and mercuric(II) iodide to form potassium tetraiodomercurate (II) (K₂[HgI₄]). The reagents which can be used to detect urea typically may be chosen from dimethylamino benzaldehyde or dimethyamino cinnamaldehyde. The kit may further contain a pH sensitive reagent which after detection of nitrate ion, can discriminate between alkali nitrates, an explosive precursor also used as fertilizer, and urea nitrate, a man made explosive.

The test kits also include a supply of one or more absorption media for sampling a suspected source of explosives. The “absorption media” may also be referred to herein without attempting to differentiate the terms as “absorption transfer media”. These may be filter paper, polyethylene laminated paper, filter paper coated with an acrylate glue to enhance sample collection, and any one of the above impregnated with at least one basic compound to neutralize the high acidity of reagent B. Basic compounds that may be used include sodium carbonate, sodium phosphate, potassium phosphate and sodium borate. This list is not meant to be limiting but exemplary only. The base used should be one that will not reduce the acid concentration of the acidified amine when the amine is placed upon it before reaction of the acid with the sample is complete. Neutralization of the acid should only occur after completion of the reaction. The suspected source of explosives may be obtained by contacting the absorption medium to a suspected substance, a surface of an inanimate object or the skin of a human.

The test kits of the present invention also include one or more, typically a plurality of, devices for dispensing the reagents in the kit. Without limiting the invention the one or more devices may be spray dispensing devices, dropper devices or breakable ampoules.

In a preferred embodiment of the kit of the invention, each of the reagents are separately contained in closed plastic dropper bottles adapted for dispensing each of the components in a dropwise manner. Also, in a particular embodiment, every dropper bottle and cap pair is identified by a color mark which is different from every other dropper bottle and cap pair in the kit, thus enabling the user to match the correct bottle and cap.

Alternatively, the reagents in the kit may be kept in crushable breakable ampoules which may be broken when the reagent is needed.

It is envisioned that the test kit taught in the present invention may be usable in the field far removed from a laboratory.

The following is a description of a method of the present invention to detect explosives containing perchlorates, chlorates, bromates, peroxides, nitrates, nitro aromatics, nitro amines and nitro esters.

The following procedures are two detecting schemes that can be used with the field test kits of the present invention.

Procedure A

A sample is collected on a collecting medium, for example, filter paper or polyethylene laminated paper or either filter paper or polyethylene laminated paper printed with a layer of acrylate glue to enhance collection of sample particles. When a drop of reagent A is placed on the sample on the collecting medium, the presence of perchlorates is indicated by the reagent's blue color changing to pink/purple. In the absence of any color change, reagent B is placed on the same paper. Alternatively, another sample collected on a new collection paper may be used for this stage. The presence of chlorates or bromates is indicated by a deep blue color. In the absence of a color change, a drop of reagent C is placed on the same paper. Development of a blue color at this stage will indicate the presence of an organic peroxide in the original sample. In the absence of any color change, a drop of reagent D is placed on the same paper. A change in color indicates the presence of nitrates.

When using high acid concentrations and/or low alcohol concentrations, nitrates react with reagent B, and their presence can not be differentiated from chlorates. However, with low acid concentrations and/or high alcohol concentrations, nitrates react with reagent D differentiating them from chlorates.

The procedure described above enables the operator to improve detection capability, by using only one collecting medium for group tests carried out sequentially. Alternatively, of course, separate samples, each on a different collecting medium, may be used for each reagent.

Procedure B

A sample is collected on a collecting medium, for example filter paper or polyethylene laminated paper or either filter paper or polyethylene laminated paper printed with a layer of acrylate glue to enhance collection of sample particles. When a drop of reagent A is placed on the sample on the collecting medium, the presence of perchlorates is indicated by the reagent's blue color changing to pink/purple. In the absence of any color change, a drop of reagent E is placed on the same paper. Reagent E forms a colored Meisenheimer complex with nitro aromatic compounds. Alternatively, a new collection paper may be used for this Meisenheimer complex stage. The presence of nitro aromatics is indicated by a purple for trinitrotoluene (TNT), yellow for picric acid, orange for N-methyl-N,2,4,6-tetranitroaniline (Tetryl), etc. In the absence of appearance of color, a drop of Griess reagent is placed on the same paper. Development of a pink-red color at this stage will indicate the presence of nitro esters or nitro amines in the sample. In the absence of any color change, a small amount of zinc dust or a drop of zinc dust suspended in an organic solution (reagent D) is placed on the same paper. A change in color indicates the presence of nitrates.

A typical Griess reagent contains 0.2% napthylenediamine dihydrochloride and 2% sulphanilamide in 5% phosphoric acid. A Griess reagent kit is readily available commercially from, for example, Promega Corporation, Madison, Wis.

In another version of Procedure B discussed herein above, another procedure hereinafter denoted as Procedure B′ is described. In procedure B′ all the steps of Procedure B are identical except that:

1. instead of using a Griess reagent to detect nitro amines or nitro esters, a drop of highly acidified carbocyclic aromatic amine solution may be used. A typical such amine is diphenylamine but other carbocyclic amines may also be used. This highly acidified amine is herein designated as reagent B, the preparation of which has been described above. Typically, acidification may be carried out using concentrated 10% or more sulfuric or phosphoric acid; and

2. if desired, after the step to determine the presence of nitro amines or nitro esters, a step to determine the presence of organic peroxides can be carried out. If after the nitro amine/nitro ester detection test there is an absence of a color change, a drop of reagent C is placed on the same paper. Development of a blue color at this stage will indicate the presence of an organic peroxide in the sample.

It should be noted that in Procedure B and B′, the use of a strongly basic solution on a sample in the step before the contacting of the sample with the amine/acid solution of reagent B is a sequence that allows the detection of nitro amines/nitro esters/nitro aromatics/chlorates/bromates and nitrates in only two steps. Such a quick detection scheme is believed to be unique.

If in Procedures A, B or B′ the presence of nitrate has been detected, a sample of the suspect substance may be tested to determine if the nitrate is an inorganic nitrate, such as an alkali nitrate, or an amine nitrate. The test is performed by using any conventional pH indicator which changes color below a pH of 5. A typical such indicator is methyl orange but it should be readily understood that this is a non-limiting example only.

It should readily be understood that, at times, Procedure B (or Procedure B′) described above may be performed before Procedure A or vice versa.

In the methods and test kits of the invention, it should readily be understood that cations other then ferric ion as discussed in reagent C can be used to detect organic peroxides. In addition to iron cations, cations of the following transition metals may be used: copper, manganese, chromium, cobalt and ruthenium. Cations from a mixture of these metals may also be used.

In the absence of a positive coloration indicating the presence of perchlorates, chlorate, bromate, peroxide and nitrates after application of Procedure A described above, the method includes preferably providing a second sample of the suspect substance, and testing it for at least one ingredient selected from nitro aromatics, nitro amines, and nitro esters according to Procedure B or B′ also described above. The reagents in the kits for applying Procedures B and B′ include the reagents adapted for performing the detection and identification tests discussed above.

It is readily understood that the field test kits taught in the present invention may include all the reagents discussed above or any subset of the reagents as desired and appropriate for the needs of the user. The exact reagents in the kit may be determined by the expected occurrence of certain classes of explosives or their expected non-occurrence.

Typical test kits may include:

Kit A comprised of:

a first reagent comprised of methylene blue solution,

a second reagent comprised of at least one aromatic amine in a strongly acidified solution;

a third reagent comprising a solution of cations of at least one transition metal; and

a fourth reagent comprising zinc powder suspended in a liquid.

Kit A′ may include Kit A and may further include Nessler's reagent for detecting urea and/or a pH sensitive reagent for discriminating between alkali nitrate salts and amine nitrates.

Kit A″ may include Kit A and further include a strongly basic solution, typically a solution of an alkali hydroxide or an alkyl ammonium hydroxide. Alternatively, Kit A″ may include Kit A wherein the methylene blue solution itself is dissolved in a strongly basic solution comprising alkali hydroxide or an alkyl ammonium hydroxide.

Kit B comprised of:

a reagent comprising methylene blue.

Kit C comprised of:

a reagent containing methylene blue for detecting a perchlorate; and

a reagent for forming a Meisenheimer complex with nitro aromatic compounds.

Kit C′ may include Kit C and further include a strongly basic solution typically an alkali hydroxide or an alkyl ammonium hydroxide solution. Alternatively, Kit C′ may include Kit C wherein the methylene blue solution itself is dissolved in a strongly basic solution comprising alkali hydroxide or an alkyl ammonium hydroxide.

Kit D comprised of:

a first reagent comprised of methylene blue solution; and

a second reagent comprised of at least one aromatic amine in a strongly acidic solution.

Kit D′ may include Kit D and further include a strongly basic solution typically an alkali hydroxide or an alkyl ammonium hydroxide solution. Alternatively, Kit D′ may include Kit D wherein the methylene blue solution itself is dissolved in a strongly basic solution comprising an alkali hydroxide or an alkyl ammonium hydroxide.

Kit E comprised of:

a first reagent containing methylene blue;

a second reagent comprising a strongly acidic solution further comprising at least one aromatic amine;

a third reagent comprising a solution of cations of at least one transition metal; and

a fourth reagent containing powdered zinc preferably suspended in a liquid.

Kit E′ may include Kit E and further include a strongly basic solution typically an alkali hydroxide or an alkyl ammonium hydroxide solution. Alternatively, Kit E′ may include Kit E wherein the methylene blue solution itself is a strongly basic solution as a result of the addition of alkali hydroxide or an alkyl ammonium hydroxide.

Kit E″ may include Kit E and may further include Nessler's reagent for detecting urea and/or a pH sensitive reagent for discriminating between alkali nitrate salts and amine nitrates.

Kit F comprised of:

a highly acidified aromatic amine solution to react with nitrates; and

a pH sensitive reagent to discriminate between alkali nitrates and amine nitrates, the former being essentially neutral and the latter being acidic.

Preparation of the reagents for the above described kits has been previously described herein above. Other reagents such as pH indicators in the acidic pH range are readily commercially available.

As noted above, it would readily be apparent that other kits may be formed of permutations and combinations of the reagents discussed herein. The kits' reagent contents are determined on the basis of the expected occurrence of an explosive class in a suspected substance.

In all of the above kits there will be included at least one device for dispensing the reagents. This at least one device may be selected from a spray device, a dropper device, or breakable ampoules.

All of the above kits will include a supply of absorbing media, or other collection or sampling means for use in sampling a suspected substance as described herein. However, it is readily evident that dedicated sampling means are not required; the reagents can be directly applied to a sample.

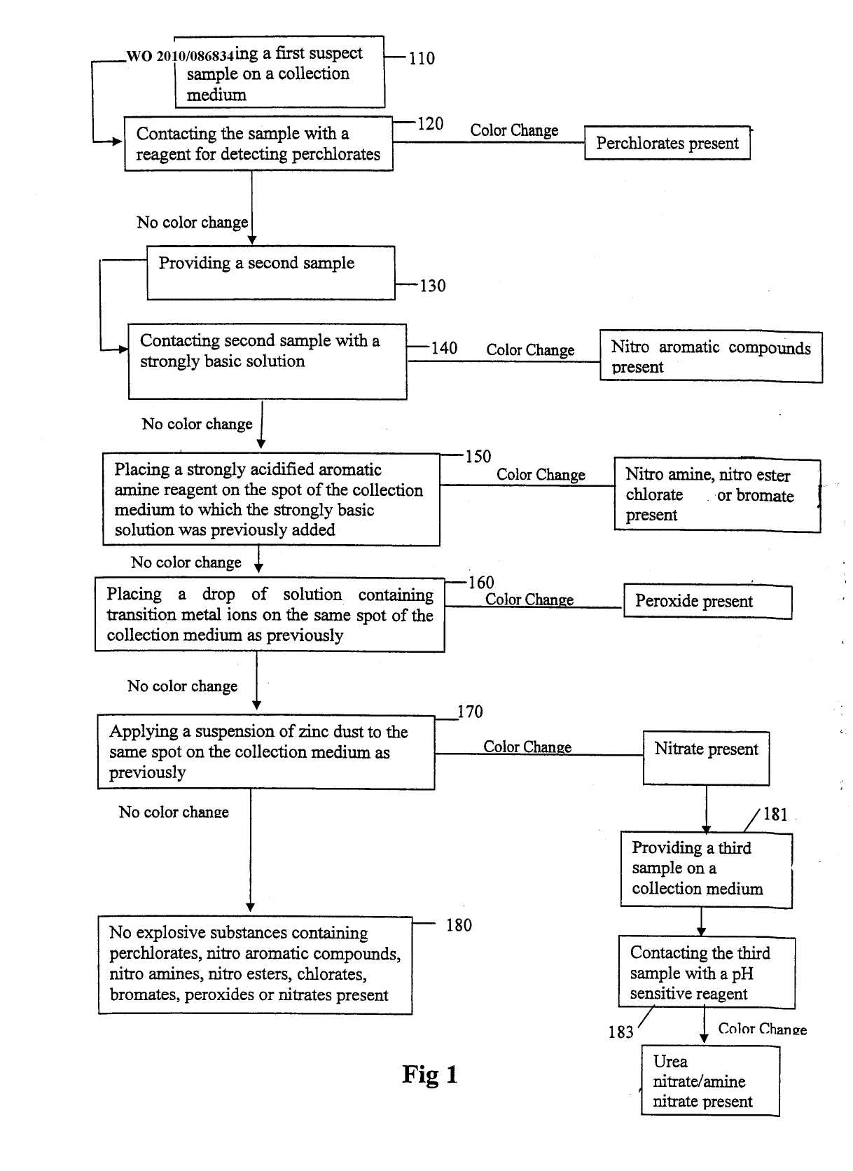

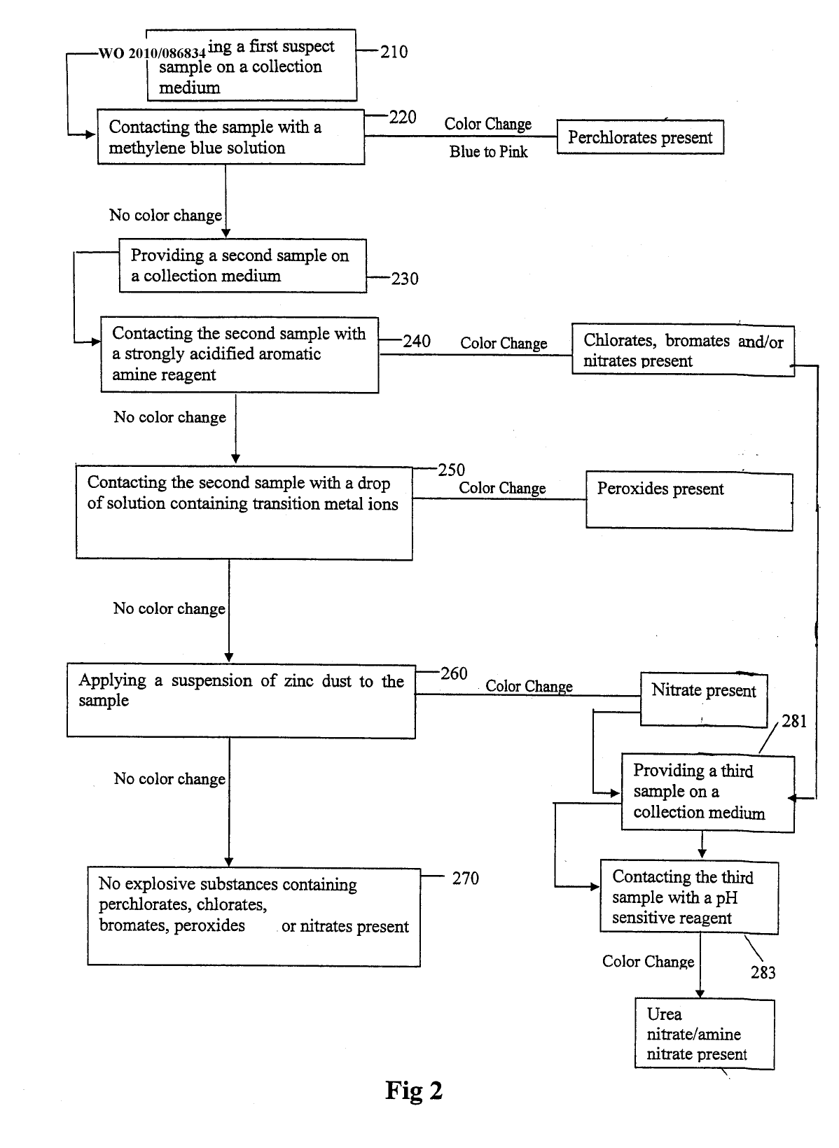

Reference is now made to FIGS. 1 and 2, in which flowcharts are presented which show the steps in Methods A and B with the additional step of Method D. The methods taught by the present invention are as follows.

Method A

A method for detecting and identifying explosives in a suspect substance, said method comprising the steps of:

providing 110 a sample of the suspect substance on a collection medium;

contacting 120 the sample with a methylene blue solution for the detection of perchlorate, wherein if perchlorate is present the solution changes color;

if no color change occurs, providing 130 a second sample and contacting 140 the second sample with a strongly basic solution wherein the base can be an alkali hydroxide or an alkylammonium hydroxide, the basic solution forming in the presence of aromatic nitro compounds a colored Meisenheimer complex having an identifiable color;

placing 150 a portion of a highly acidified solution of an aromatic amine on substantially the same spot of the absorbent medium as in the previous steps, the aromatic amine being oxidized by chlorates, bromates and nitrites, the latter released by the basic hydrolysis of nitro amines or nitro esters occurring in the second step of contacting, thereby producing a change in color identifying the presence of nitro amines, nitro esters, chlorates and bromates in the sample;

when no reaction from the steps of contacting and the step of placing has occurred, placing 160 a drop of solution containing transition metal cations in a non-aqueous solvent on the same spot of the absorbent medium as in the previous steps and if peroxides are present, the cations catalyze oxidation of the aromatic amine to produce a change in color; and

when no color change has occurred in any of the previous steps, applying 170 a suspension of zinc dust to the same spot of the absorbent medium that under the acidic conditions still present reduced any nitrate salts present to nitrites that in turn now oxidize the aromatic amine causing the reagents to turn blue.

A flowchart of Method A is shown as FIG. 1.

Method B

A method for detecting and identifying explosives in a suspect substance, further including the steps of:

providing 210 a sample of a substance suspected of being an explosive;

contacting 220 the sample with a first reagent, the first reagent being a solution of methylene blue which turns pink in the presence of perchlorates;

providing 230 a second sample and contacting 240 the second sample with a second reagent, the second reagent comprising at least one aromatic amine in a strongly acidic solution, whereby the presence of a chlorate, bromate, or nitrate explosive produces a distinct coloration; and

in the absence of such distinct coloration, contacting 250 the same sample, which has already been in contact with the second reagent, with a third reagent, the third reagent comprising a solution of cations of at least one transition metal, whereby the presence of peroxides in the sample, which have been hydrolyzed at least partly to hydrogen peroxide by the strong acid of the second reagent, produces a distinct coloration; and

in the absence of any coloration, applying 260 to the same sample, which is already in contact with the first, second and third reagents, a fourth reagent, the fourth reagent comprising powdered zinc suspended in a liquid, and the fourth reagent producing a distinct blue color when nitrates are present.

A flowchart of Method B is shown in FIG. 2 herein.

Method C

Method A followed by Method B or Method B followed by Method A.

Method D

If nitrates have previously been detected in either Method A or B the following further steps are included:

providing 181, 281 a third sample of the suspect substance on an absorbent medium; and

contacting 183, 283 the suspected explosive with a pH sensitive reagent for discriminating between alkali nitrate salts and amine nitrate salts and noting the pH, low pH indicating the possible presence of an explosive amine nitrate.

Method D appears in FIGS. 1 and 2 herein.

Method E

A method for detection of perchlorate, nitro aromatic, nitro ester and nitrate substances in a suspect substance, said method comprising the steps of:

providing a sample of the suspect substance on an absorbent medium;

contacting the sample with a solution for the detection of perchlorate wherein if perchlorate is present the solution changes color;

contacting a sample with a strongly basic solution, wherein the base can be selected from an alkali hydroxide or an alkylammonium hydroxide, the basic solution forming in the presence of nitro aromatic compounds a colored Meisenheimer complex having an identifiable color change and hydrolyzing the nitro amines or nitro esters to nitrites if nitro amines or nitro esters are present;

placing a portion of a strongly acidified solution of an aromatic amine on the same spot of the absorbent medium so that any nitrites present will oxidize the aromatic amine producing a change in color.

Method F

A method for detection of perchlorate, nitro aromatic, nitro ester, nitro amine and nitrate salts, said method including the steps of:

providing a sample of a suspect substance;

contacting the sample with a reagent for detecting perchlorates;

when there is no change in color in said step of contacting, contacting the same sample with a reagent for forming a colored Meisenheimer complex with a nitro aromatic the reagent being a highly alkaline solution;

when there is no reaction in the second step of contacting, contacting the same sample with a reagent that reacts with nitro esters and nitro amines; and

when there is no reaction, contacting the same sample with a reagent that reacts with nitrates.

Method G

A method for detection and identification of oxidant materials comprising:

contacting a first sample with a solution of methylene blue to produce a color change in the presence of perchlorates; and

reacting a second sample with a highly acidified solution of an aromatic amine to produce a color change in the presence of oxidants selected from the group of chlorates, bromates, permanganates, nitrates and chromates.

It should also be remembered that other oxidants not normally thought of as explosives, such as permanganates and chromates, may also be detected by the reagents, kits and methods of the present invention. For example, permanganates and chromates may react with reagent B discussed above each producing a distinctive color. Accordingly, oxidants other than those specifically noted herein are also contemplated as being within the metes and bounds of the present invention.

Method H

A method for discriminating between alkali nitrates and amine nitrates comprising the steps of:

contacting a first sample with a reagent for determining the presence of nitrate; and

if nitrate is found, contacting a second sample with a pH indicator that changes color below a ph of 5.

It should readily be understood by one skilled in the art that in all the multi-step methods presented herein above, the method may be truncated once a reagent produces a color reaction without carrying out the remaining steps. This effectively allows the step employing the reagent producing the color change and all reagents employed in previous steps without producing a color change to be considered to be a method in itself.

It is to be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention. Therefore, it will be appreciated by persons skilled in the art that the present invention is not limited by what has been particularly shown and described herein above. Rather, the scope of the invention is defined by the claims that follow.

1. A test kit for detecting oxidants in a sample suspected of being an explosive, said kit comprising:
a. a methylene blue solution for detecting perchlorates;
b. one or more aromatic amine in a strongly acidic solution for detecting chlorates. bromates and/or nitrates;
c. a strongly basic solution for forming a colored Meisenheimer complex with a nitro aromatic compound and for hydrolyzing nitro esters and nitro amines to produce nitrites;
d. an absorbent medium for absorbing said methylene blue solution enhancing visibility of any color change.

2. (canceled)

3. (canceled)

4. A kit according to claim 1 wherein said methylene blue is in a water solvent solution with a preferred concentration range of about 0.01% to about 0.2% w/w and said solvent is an alcohol preferably in a concentration range of about 10% to about 30% v/v alcohol to water.

5. (canceled)

6. (canceled)

7. (canceled)

8. A kit according to claim 1 wherein said reagent for forming a colored Meisenheimer complex is a strongly basic reagent, said strongly basic reagent being said methylene blue solution which further comprises a basic compound selected from a group consisting of an alkyl ammonium hydroxide, sodium hydroxide and potassium hydroxide.

9. A kit according to claim 1 further comprising:
a solution of cations of a transition metal for detecting peroxides; and
a reagent comprising zinc powder suspended in a liquid for detecting nitrates.

10. A kit according to claim 9, further comprising a pH sensitive reagent for discriminating between alkali nitrate salts and amine nitrates, and wherein said pH sensitive reagent is a pH indicator that changes color under pH 5.

11. (canceled)

12. A kit according to claim 9, which is further characterized by one or more of the following features:
(a) one or more of said methylene blue and said acidified aromatic amine reagents include one or more water-miscible non-aqueous solvents;
(b) said aromatic amine comprises a carbocyclic secondary aromatic amine;
(c) said strongly acidic solution of said aromatic amine reagent comprises sulfuric acid or phosphoric acid;
(d) said cations of said transition metal are cations selected from a group consisting of iron, copper, manganese, chromium, cobalt and ruthenium cations;
(e) said absorbent medium of said kit is impregnated with a basic substance to neutralize excess acid in said acidified aromatic amine wherein said basic substance is selected from a group consisting of sodium carbonate, sodium phosphate, potassium phosphate and sodium borate;
(f) said kit comprises an additional reagent adapted for testing for the presence of urea and nitro amines; and
(g) said kit comprises a reagent which is a strongly basic solution comprising an alkali hydroxide or an alkyl ammonium hydroxide.

13. (canceled)

14. A method for detecting and identifying oxidants in a substance suspected of being an explosive, said method including the steps of:
providing a sample of the substance suspected of being an explosive;
contacting the sample with a first reagent, the first reagent being a solution for the detection of perchlorate, wherein if perchlorate is present the solution changes color; and
contacting the same sample with a second reagent, the second reagent comprising one or more aromatic amines in a strongly acidic solution, whereby the presence of a chlorate, bromate or nitrate explosive produces a distinctive coloration.

15. (canceled)

16. (canceled)

17. A method according to claim 14, said method further comprising the steps of:
if no color change has occurred in any of the previous steps of the method, contacting a sample with a strongly basic solution, wherein the base can be an alkali hydroxide or alkylammonium hydroxide, the basic solution forming in the presence of aromatic nitro compounds a colored Meisenheimer complex having an identifiable color change and if there are nitro amines or nitro esters present the basic solution hydrolyzes the nitro amines or nitro esters to nitrites; and
if no color change has occurred in any of the previous steps of the method, placing a drop of the second reagent of an aromatic amine on the same spot of the absorbent medium that was contacted previously so that any nitrites present will oxidize the aromatic amine thereby producing a change in color.

18. (canceled)

19.-23. (canceled)

24. A method according to claim 14, wherein the reagent for detecting perchlorates is methylene blue.

25.-29. (canceled)

30. A method according to any one of claims 14 wherein when nitrates are detected said method further includes the step of:
contacting the suspected explosive with a pH sensitive reagent for discriminating between alkali nitrate salts and amine nitrate salts by noting the pH, low pH indicating the possible presence of an explosive amine nitrate.

31. (canceled)

32. A reagent for the detection of perchlorate said reagent comprising methylene blue.

33. A reagent for the detection of nitro esters and nitro amines, said reagent comprising a strongly acidic solution of an aromatic amine, wherein said reagent produces a change in color after reacting with nitrites resulting from the hydrolysis of the nitro amines and nitro esters being exposed to a strongly basic solution.

34.-45. (canceled)

46. A method according to any one of claims 17 wherein when nitrates are detected said method further includes the step of:
contacting the suspected explosive with a pH sensitive reagent for discriminating between alkali nitrate salts and amine nitrate salts by noting the pH, low pH indicating the possible presence of an explosive amine nitrate.
